(19) United States
(12) Patent Application Publication
(10) Pub. No.: US 2019/0239749 A1
(43) Pub. Date: Aug. 8, 2019

(54) IMAGING APPARATUS

(71) Applicant: Shimadzu Corporation, Kyoto (JP)

(72) Inventor: Akihiro ISHIKAWA, Kyoto (JP)

(73) Assignee: Shimadzu Corporation, Kyoto (JP)

(21) Appl. No.: 15/891,705

(22) Filed: Feb. 8, 2018

(51) Int. Cl. A61B 5/00; A61K 49/00

(52) U.S. Cl. CPC A61B 5/0071; A61K 49/0034

(57) ABSTRACT

A control unit is connected to an illuminating/photographing unit including a camera, an infrared light source and a visible light source, and a storage unit including an image storage unit. The control unit includes an image processing unit. The image processing unit includes a combining unit that combines a fluorescence image acquired by the illuminating/photographing unit and a visible light image, a determination unit that determines whether division in running of the blood vessel exists or not, and a coloring unit that performs coloring of a different color for each blood vessel area determined to be divided at the determination unit with respect to the blood vessel.

### FIELD

The present invention relates to an imaging apparatus that irradiates a fluorescence substance infiltrated into a body of a subject with excitation light and photographs fluorescence that is emitted from the fluorescence substance.

### BACKGROUND

A technique called near-infrared fluorescence imaging is used for angiography in surgical operation. In this near-infrared fluorescence imaging, indocyanine green (ICG) which is a fluorescence dye is injected into an affected part. When the indocyanine green is irradiated with near-infrared light having a wavelength of about 810 nm (nanometer) as excitation light, indocyanine green emits near-infrared fluorescence having a wavelength of approximately 845 nm. The fluorescence is photographed by an imaging element capable of detecting the near-infrared light, and the image is displayed on a display unit of a liquid crystal display panel or the like. According to the near-infrared fluorescence imaging, blood vessels, lymphatic vessels, and the like existing at a depth of about 20 mm from a body surface may be observed.

Further, in recent years, a method of fluorescently labeling a tumor and using it for surgical navigation has attracted attention. As a fluorescence labeling agent for fluorescently labeling the tumor, 5-aminolevulinic acid (5-ALA) is used. When the 5-aminolevulinic acid (hereinafter, referred to as “5-ALA” when abbreviated) is administrated to the subject, the 5-ALA is metabolized to PpIX (protoporphyrinlX/protoporphyrin nine) which is a fluorescence substance. The PpIX is accumulated in cancer cells specifically. When the PpIX which is a metabolite of 5-ALA is irradiated with visible light having a wavelength of about 410 nm, red visible light having a wavelength of about 630 nm is emitted as fluorescence from the PpIX. By observing the fluorescence from this PpIX, cancer cells can be confirmed.

International Publication No. 2009/139466 discloses a data collection method in which an intensity distribution image of near-infrared fluorescence obtained by irradiating a test organ of a living body to which the indocyanine green has been administered with excitation light of the indocyanine green and a cancer lesion distribution image obtained by applying X-ray, nuclear magnetic resonance or ultrasonic wave to the test organ before the indocyanine green is administrated are compared, and data of an area which is detected in the intensity distribution image of the near-infrared fluorescence but is not detected in the cancer lesion distribution image is collected as secondary cancer lesion area data.

### SUMMARY

In such an imaging apparatus for photographing the fluorescence from the fluorescence substance infiltrated into the body, a single camera photographs visible light and near-infrared light at the same time, and a photographed image recorded by a video recorder is reproduced as a moving image. In order to facilitate an observation of running of a blood vessel and a lymphatic vessel after the ICG is administrated under a bright external illumination environment, coloring of a near-infrared fluorescence detection area of the image has been performed. In the related art, since the fluorescence detection area in the image is colored with a single color, an operator determines that the running of the blood vessel is divided or stenosis of the lymphatic vessel is present, when detecting a discontinuous part of the blood vessel or the like expressed in the same color from the image.

It may take time for the operator to detect the discontinuous part of the same colored blood vessel and the like in the image. Particularly, in the case of performing angiography by irradiating an organ with an infrared light during a surgical operation, it is not preferable, from the viewpoint of a burden on a patient, that it takes time to determine the division in the running of the blood vessel, so that an operation time becomes long.

The present invention was made to solve the aforementioned problem, and intends to provide an imaging apparatus capable of assisting an operator to determine division in running of a blood vessel or stenosis of a lymphatic vessel.

The present invention includes: an excitation light source that irradiates a fluorescence substance infiltrated into a body of a subject with an excitation light; a photographing unit that detects and photographs a fluorescence excited by the excitation light and generated from the fluorescence substance; an image processing unit that displays a fluorescence image acquired by photographing the fluorescence with the use of the photographing unit on a display unit; and an image storage unit that stores the fluorescence image. The image processing unit includes a determination unit that identifies continuous areas in which the fluorescence in the fluorescence image is detected and a coloring unit that performs a coloring by designating a different color for each of the continuous areas determined in the determination unit, and the fluorescence image colored with the color designated for each of the continuous areas by the coloring unit is displayed on the display unit.

The present invention may further include: an excitation light source that irradiates a fluorescence substance infiltrated into a body of a subject with excitation light; a visible light source that irradiates the subject with white light; a photographing unit that detects and photographs a fluorescence excited by the excitation light and generated from the fluorescence substance and a reflected light of the white light; an image processing unit including a combining unit that creates a combined image obtained by combining a fluorescence image acquired by photographing the fluorescence and a visible light image acquired by photographing the reflected light with the use of the photographing unit; and an image storage unit that stores the fluorescence image, the visible light image and the combined image. The image processing unit includes a determination unit that identifies continuous areas in which the fluorescence in the fluorescence image is detected and a coloring unit that performs a coloring by designating a different color for each of the continuous areas determined in the determination unit, and the visible light image colored with the color designated for each of areas corresponding to the continuous areas by the coloring unit or the combined image colored with the color designated for each of areas corresponding to the continuous areas by the coloring unit are displayed on the display unit.

### Advantageous Effects of the Invention

According to the present inventions, since there is provided the image processing unit including the determination unit that identifies continuous areas in which the fluorescence in the fluorescence image is detected and the coloring unit that performs the coloring by designating the different color for each of the continuous areas determined in the determination unit, it is possible to display on the display unit an image obtained by coloring a different color for each divided blood vessel or for each divided lymphatic vessel. This makes it possible to support determination of the operator with respect to the division in running of the blood vessel and the stenosis of the lymphatic vessel.

### BRIEF DESCRIPTION OF THE DRAWINGS

FIG. 1 is a schematic diagram of an imaging apparatus according to the present invention;

FIG. 2 is a schematic diagram of an illuminating/photographing unit 12;

FIG. 3 is a block diagram illustrating a main control system of an imaging apparatus according to the present invention;

FIG. 4 is a schematic diagram illustrating an example of a display mode of each image on a display unit 14; and

FIG. 5A is a schematic diagram illustrating a first image displayed on the display unit 14.

FIG. 5B is a schematic diagram illustrating a second image displayed on the display unit 14.

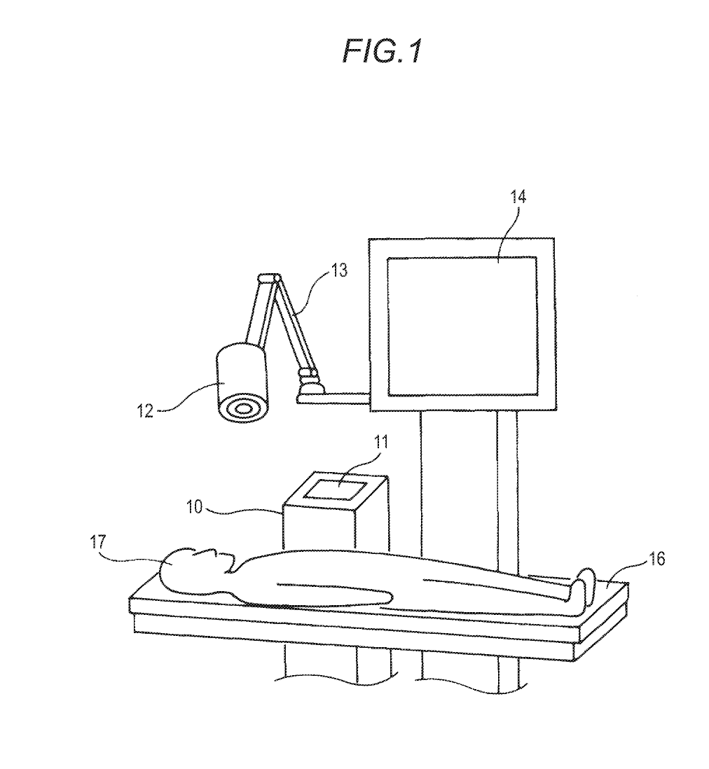

Hereinafter, embodiments of the present invention will be described with reference to the drawings. FIG. 1 is a schematic diagram of an imaging apparatus according to the present invention.

The imaging apparatus includes a main body 10 including an input unit 11 such as a touch panel and having a control unit 30, a storage unit 40 and the like to be described later incorporated therein, an illuminating/photographing unit 12 movably supported by an arm 13, a display unit 14 including a liquid crystal display panel and the like, and a treatment table 16 on which a patient 17 is placed. The illuminating/photographing unit 12 is not limited to the one supported by the arm 13, but may be carried by an operator in hand or fixed to an existing facility.

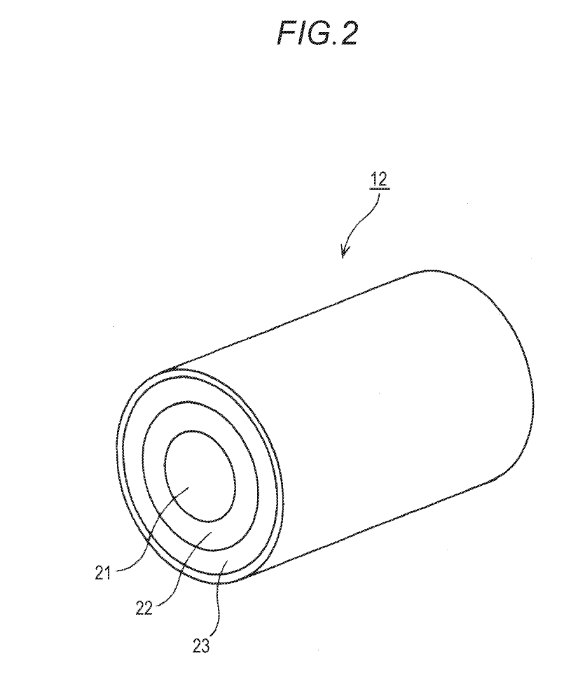

FIG. 2 is a perspective view of the illuminating/photographing unit 12.

The illuminating/photographing unit 12 includes a camera 21 capable of detecting near-infrared light and visible light, an infrared light source 22 disposed on an outer periphery of the camera 21, and a visible light source 23 disposed on an outer periphery of the infrared light source 22. The infrared light source 22 is an excitation light source that excites a fluorescence substance that has infiltrated into a body of the patient 17. Further, the visible light source 23 irradiates the patient 17 with white light.

In the present embodiment, the illuminating/photographing unit 12 integrating the infrared light source 22 and the visible light source 23 and the camera 21 is used, but the infrared light source 22, the visible light source 23, and the camera 21 may be individually disposed. When only the fluorescence image is displayed on the display unit 14, the visible light source 23 may not be provided.

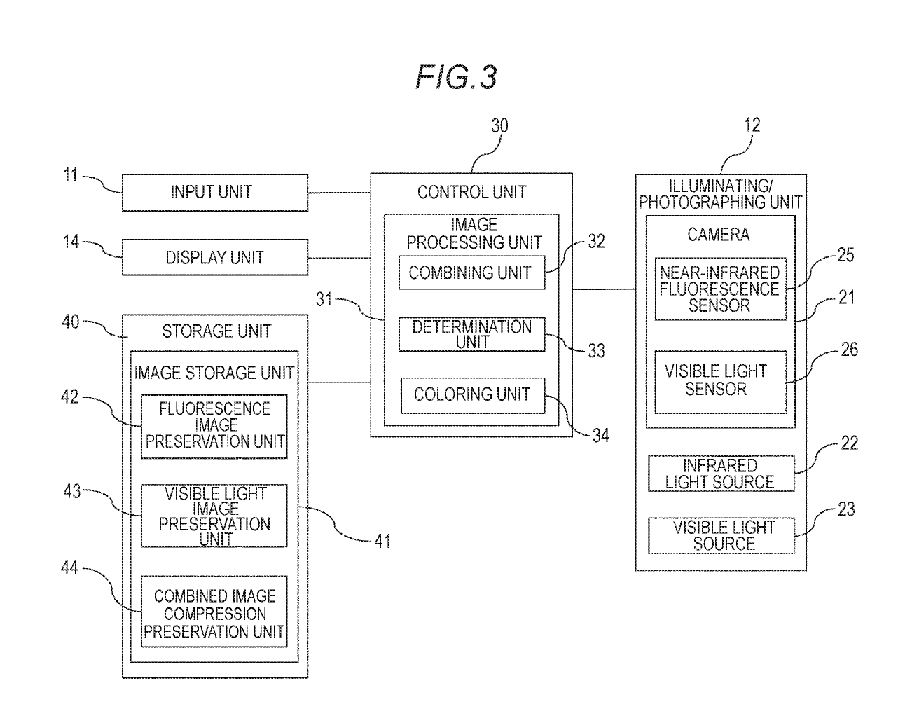

FIG. 3 is a block diagram illustrating a main control system of the imaging apparatus according to the present invention.

The imaging apparatus includes a CPU for executing a logical operation, a ROM in which operation programs necessary for controlling the apparatus are stored, a RAM in which data and the like are temporarily stored during control, and the like, and includes a control unit 30 for controlling the entire apparatus. The control unit 30 is connected to the input unit 11 and the display unit 14 described above.

Further, the control unit 30 is connected to the illuminating/photographing unit 12 including the camera 21, the infrared light source 22, and the visible light source 23. The camera 21 includes a near-infrared fluorescence sensor 25 as an image sensor for detecting near-infrared fluorescence and a visible light sensor 26 as an image sensor for detecting reflected light (visible light) of the white light. The fluorescence and the visible light incident on the camera 21 are separated by a spectroscopic mechanism inside the camera 21 and detected by each image sensor. Then, a fluorescence image and a visible light image detected by each image sensor are sent to the control unit 30. Since the camera 21 includes the near-infrared fluorescence sensor 25 and the visible light sensor 26, the imaging apparatus may acquire the fluorescence image and the visible light image synchronously in the same field of vision.

The control unit 30 includes an image processing unit 31. The image processing unit 31 includes a combining unit 32 that combines the fluorescence image acquired by the illuminating/photographing unit 12 and the visible light image, a determination unit 33 that determines whether division in running of the blood vessel exists or not, and a coloring unit 34 that performs coloring of different colors for each blood vessel area determined to be divided at the determination unit 33 with respect to the blood vessel.

Further, the control unit 30 is connected to a storage unit 40 that stores the image and the like photographed by the camera 21. The storage unit 40 includes an image storage unit 41 including a fluorescence image preservation unit 42 that preserves the fluorescence image, a visible light image preservation unit 43 that preserves the visible light image, and a combined image compression preservation unit 44 that compresses and preserves the combined image obtained by combining the fluorescence image and the visible light image at the combining unit 32 of the image processing unit 31.

Hereinafter, an operation in a surgical operation using the imaging apparatus according to the present invention will be described. The case of performing a fluorescence angiography on the patient 17 during an operation will be described as an example.

In the case of performing a fluorescence angiography using the imaging apparatus according to the present invention during a surgical operation, indocyanine green is injected by injection into the patient 17 lying on the treatment table 16. Then, the subject including an affected part is irradiated with infrared light emitted from the infrared light source 22 and white light emitted from the visible light source 23. As the infrared light, the near-infrared light of 750 to 850 nm used as excitation light that causes the indocyanine green to emit fluorescence is adopted. Thus, the indocyanine green generates fluorescence of a near-infrared area having a peak at 845 nm.

Then, the vicinity of the affected part of the patient 17 is photographed by the camera 21. The camera 21 is capable of detecting an infrared light and a visible light. The fluorescence image and the visible light image photographed by the camera 21 are sent to the image processing unit 31 illustrated in FIG. 3. The image processing unit 31 converts the fluorescence image and the visible image into image data that can be displayed on the display unit 14. That is, the fluorescence image is converted into 8-bit image data, and the visible light image is converted into 24-bit image data formed of three colors of RGB. The data of the fluorescence image is preserved in the fluorescence image preservation unit 42 of the image storage unit 41. In addition, the data of the visible light image is preserved in the visible light image preservation unit 43 of the image storage unit 41.

The determination unit 33 analyzes the 8-bit image data of the fluorescence image on a pixel-by-pixel basis to determine whether the division in the running of the blood vessel exists or not. For example, whether the division in the running of the blood vessel exists or not is determined based on whether pixel values in a predetermined range of the image data of the fluorescence image are continuous or not. Then, the determination unit 33 extracts continuous areas of the pixel values in the predetermined range as one blood vessel area. Thereafter, the coloring unit 34 refers to a color table stored in the storage unit 40 for each blood vessel area extracted by the determination unit 33, and designates a color for each area.

The combining unit 32 in the image processing unit 31 creates a combined image acquired by merging the fluorescence image and the visible light image using fluorescence image data and visible light image data. The combined image is stored as a moving-image reproduction image in the combined image compression preservation unit 44 in the image storage unit 41 of the storage unit 40. Then, the image processing unit 31 displays the infrared image, the visible light image, and the combined image simultaneously or selectively on the display unit 14.

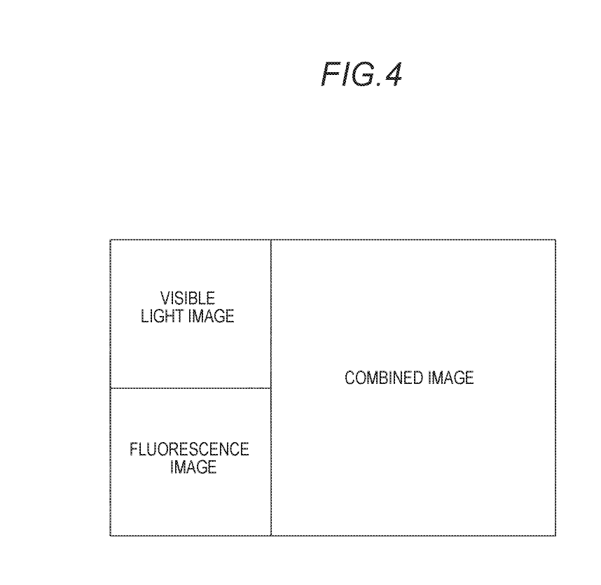

FIG. 4 is a schematic diagram illustrating an example of a display mode of each image on the display unit 14.

In the case of simultaneously displaying the fluorescence image, the visible light image and the combined image on the display unit 14, display areas of the respective images are provided on a screen of the display unit 14, as illustrated in FIG. 4. The image processing unit 31 captures the fluorescence image and the visible light image photographed by the camera 21, merges them in the combining unit 32, and displays the combined image near the affected area of the patient 17 on the display unit 14 as a moving image. Further, the image processing unit 31 reads out the fluorescence image stored in the fluorescence image preservation unit 42 and the visible light image stored in the visible light image preservation unit 43, and displays them as still images on respective display areas provided in the display unit 14.

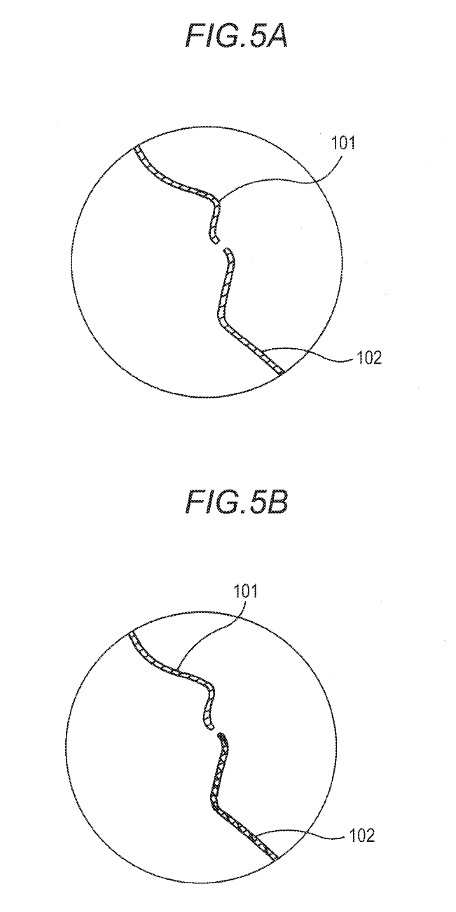

FIG. 5 is a schematic diagram illustrating an image displayed on the display unit 14.

In the image displayed on the display unit 14 illustrated in FIG. 5, blood vessels 101 and 102 where the fluorescence generated from indocyanine green is colored with a predetermined color are displayed. FIG. 5A illustrates a state in which the blood vessels 101 and 102 are colored with a conventional single color, and FIG. 5B illustrates a state where the blood vessels 101 and 102 are colored with different colors respectively. In FIG. 5, a difference between the colors used for coloring applied to blood vessels 101 and 102 respectively is indicated by different hatching.

The operator observing the display unit 14 may recognize more clearly that the blood vessel is divided when the blood vessels 101 and 102 are colored with different colors, compared to a case where the blood vessels 101 and 102 are expressed in a single color as in the related art. Thus, the operator may perform the determination of the running of the blood vessels, a bloodstream evaluation and the like more quickly. As a result, it is possible to shorten a process of checking the running of the blood vessels and the like with the use of the display unit 14 during the operation, thereby shortening an operation time.

Further, the image where the blood vessels 101 and 102 are colored with different colors respectively may be any one of the fluorescence image, the visible light image, and the combined image. For example, when the illuminating/photographing unit 12 does not include the visible light source 23 and the visible light sensor 26, only an image obtained by coloring the fluorescence image or the fluorescence image and the image obtained by coloring the fluorescence image have only to be displayed on the display unit 14. In other words, an image where different colors are colored for divided blood vessels respectively is appropriately changed, depending on a type of the image that can be acquired by the control unit 30 through a configuration of the illuminating/photographing unit 12 and a type of the image selected to be displayed on the display unit 14 in the image processing unit 31.

In FIG. 5, an example of displaying the division in the running of the blood vessel is described. However, it is possible to support determination of the operator concerning stenosis of a lymphatic vessel during the operation by displaying an image in which different coloring is performed to each of continuous regions of the lymphatic vessel on the display unit 14.

Further, in the aforementioned embodiment, a case where indocyanine green is used for angiography of the patient 17 has been described. However, the present invention may also be applied to the case of using other fluorescent labeling agent such as 5-ALA which is metabolized to protoporphyrin IX (PpIX) which is a fluorescent substance in a cancer cell.

1. An imaging apparatus comprising:
an excitation light source that irradiates a fluorescence substance infiltrated into a body of a subject with an excitation light;
a photographing unit that detects and photographs a fluorescence excited by the excitation light and generated from the fluorescence substance;
an image processing unit that displays a fluorescence image acquired by photographing the fluorescence with the use of the photographing unit on a display unit; and
an image storage unit that stores the fluorescence image,
wherein the image processing unit includes a determination unit that identifies continuous areas in which the fluorescence in the fluorescence image is detected and a coloring unit that performs a coloring by designating a different color for each of the continuous areas determined in the determination unit, and
wherein the fluorescence image colored with the color designated for each of the continuous areas by the coloring unit is displayed on the display unit.

2. An imaging apparatus comprising:
an excitation light source that irradiates a fluorescence substance infiltrated into a body of a subject with excitation light;
a visible light source that irradiates the subject with white light;
a photographing unit that detects and photographs a fluorescence excited by the excitation light and generated from the fluorescence substance and a reflected light of the white light;
an image processing unit including a combining unit that creates a combined image obtained by combining a fluorescence image acquired by photographing the fluorescence and a visible light image acquired by photographing the reflected light with the use of the photographing unit; and
an image storage unit that stores the fluorescence image, the visible light image and the combined image,
wherein the image processing unit includes a determination unit that identifies continuous areas in which the fluorescence in the fluorescence image is detected and a coloring unit that performs a coloring by designating a different color for each of the continuous areas determined in the determination unit, and
wherein the visible light image colored with the color designated for each of areas corresponding to the continuous areas by the coloring unit or the combined image colored with the color designated for each of areas corresponding to the continuous areas by the coloring unit is displayed on a display unit.
